(19) United States
(12) Patent Application Publication
(10) Pub. No.: US 2026/0208104 A1
(43) Pub. Date: Jul. 23, 2026

(54) MODULAR CARBON CAPTURE SYSTEM

(71) Applicant: University of Technology Sydney, Ultimo, New South Wales (AU)

(72) Inventors: Peter RALPH, Ultimo (AU); DucLong NGHIEM, Ultimo (AU); Andrei HERDEAN, Ultimo (AU); Luong Ngoc NGUYEN, Ultimo (AU); Leen LABEEUW, Ultimo (AU); Bernardo CAMPOS DIOCARETZ, Ultimo (AU)

(21) Appl. No.: 19/140,472

(22) PCT Filed: Dec. 18, 2023
(86) PCT No.: PCT/AU2023/051317

§ 371 Date: Jun. 18, 2025

(30) Foreign Application Priority Data
Dec. 19, 2022 (AU) 2022903901
Dec. 19, 2022 (AU) 2022903902
Dec. 19, 2022 (AU) 2022903903
Dec. 19, 2022 (AU) 2022903904

(51) Int. Cl. B01D 53/84; B01D 53/62; C12M 1/00; C12M 1/02; C12M 1/12; C12M 1/26; C12M 1/34; C12M 1/36

(52) U.S. Cl. CPC B01D 53/84; B01D 53/62; C12M 21/02; C12M 23/04; C12M 27/00; C12M 29/06; C12M 29/14; C12M 33/00; C12M 39/00; C12M 41/06; C12M 41/26; C12M 41/30; C12M 41/34; C12M 41/48; C12M 43/08; B01D 2257/504; B01D 2258/0283; B01D 2258/06; B01D 2259/802; B01D 2259/804

(57) ABSTRACT

The invention relates to a carbon capture system, including an illumination apparatus, the illumination apparatus, including: at least one tray, the at least one tray including a substantially planar base and a peripheral rim around the base to form a receptacle; a supporting frame configured to receive the at least one tray; a light-dispersing layer disposed on a surface of the planar base to enable substantially uniform distribution of light through the planar base; and at least one light source, positioned adjacent the planar base and configured to emit light toward the planar base such that at least a portion of the emitted light is dispersed by the light dispersing layer through the surface of the planar base. The system further includes a carbonation device, a defouler, an automated nutrient dosing system, and a harvester. The carbon capture system may also be configured to carry out a method for determining optimal temporal variations in flux output for algal growth.

This application claims the benefit of Convention priority from Australian provisional patent application nos. 2022903901, 2022903902, 2022903903 and 2022903904, filed 19 Dec. 2023, the contents of each of which are incorporated herein by reference in their entirety.

### FIELD OF THE INVENTION

The present invention relates to carbon capture system. More particularly, the invention relates to a modular algal carbon capture system.

The invention has been developed primarily for use as a modular algal carbon capture and utilisation system. However, while some embodiments will be described herein with particular reference to that application, it will be appreciated that the invention is not limited to such a field of use, and is applicable in broader contexts.

### BACKGROUND

The following discussion of the prior art is intended to facilitate an understanding of the invention and to enable the advantages of it to be more fully understood. It should be appreciated, however, that any reference to prior art throughout the specification in no way be considered as an admission that such art is widely known or forms part of the common general knowledge in the field.

Existing algal carbon capture and utilization (CCU) systems include photobioreactors which face limitations in scalability, efficient power usage and effective carbon utilization for algal growth. Algae require light energy, dissolved carbon dioxide and nutrients for their photosynthesis to generate biomass. Existing CCU systems do not effectively address the needs of the algae to ensure optimal growth and carbon capture and utilization. Slow algae growth rates are a major challenge to algal carbon capture systems because of limited light energy uptake by existing bioreactor configurations, which are typically a pond or raceway type system. Furthermore, these systems are susceptible to light loss by scattering, resulting in inefficient light and energy usage.

In current large-scale algae cultivation CO₂ supply is not configured to ensure that the carbon captured from an environment or used from a concentrated source is adequately utilized by the algae rather than being lost back to the atmosphere. CO₂ gas loss is a deficiency of the existing systems.

Existing CCU systems also require a substantial surface footprint to provide sufficient illumination and carbonation for algae growth. These systems cannot easily be scaled to increase in size and illumination area.

It is an object of the present invention to overcome or ameliorate at least one of the disadvantages of the prior art, or to provide a useful alternative.

### SUMMARY OF THE INVENTION

According to a first aspect of the invention, there is provided a carbon capture system, including:
an illumination apparatus, the illumination apparatus, including:
   at least one tray, the at least one tray including a substantially planar base and a peripheral rim around the base to form a receptacle;
   a supporting frame configured to receive the at least one tray;
   a light-dispersing layer disposed on a surface of the planar base to enable substantially uniform distribution of light through the planar base; and
   at least one light source, positioned adjacent the planar base and configured to emit light toward the planar base such that at least a portion of the emitted light is dispersed by the light dispersing layer through the surface of the planar base.

In another embodiment, the carbon capture system further includes a carbonation device, the carbonation device including:
a fluid vessel;
a fluid supply system disposed on an upper portion of the fluid vessel to supply fluid to the fluid vessel;
a gas injection inlet disposed on a lower portion of the fluid vessel to supply gas to the fluid vessel;
at least one mixing element arranged within the fluid vessel and below the fluid supply system;
a diffuser arranged within the fluid vessel such that gas supplied through the gas injection inlet passes at least partially through the diffuser to produce a diffused gas, wherein the diffused gas and fluid mix within the fluid vessel to produce a mixed fluid; and
an outlet disposed on the fluid vessel to release the mixed fluid.

In another embodiment, an assessment device in communication with the illumination apparatus is configured to carry out a method for determining optimal temporal variations in flux output for algal growth, the method including the steps of:
providing at least one sample on a sample plate;
controlling at least one secondary light source to emit light towards the sample plate in accordance with flux output conditions, wherein the flux output conditions include at least a duty cycle and a pulse frequency;
exposing the sample to the emitted light for a predefined time interval;
obtaining a first measurement of at least one response parameter related to the at least one sample after it has been exposed to the emitted light for a defined time interval;
adjusting the flux output conditions based on the at least one response parameter;
obtaining a second measurement of at least one response parameter related to the at least one sample after it has been exposed to emitted light having adjusted flux output conditions for a second defined time interval;
identifying an optimal set of flux output conditions from the at least one response parameter when the response parameter is determined to reach a threshold value;
outputting the optimal set of flux output conditions as an optimal temporal variation of flux output for the sample; and
controlling the at least one light source of the illumination apparatus in accordance with the optimal temporal variation of flux output.

In a further embodiment, the illumination apparatus is configured to carry out a method for determining optimal temporal variations in flux output for algal growth, the method including the steps of:
providing an algae solution on the at least one tray;
controlling the at least one light source of the illumination apparatus to emit light towards the at least one tray in accordance with flux output conditions, wherein the flux output conditions include at least a duty cycle and a pulse frequency;
exposing the at least one tray to the emitted light for a predefined time interval;
obtaining a first measurement of at least one response parameter related to the algae solution after it has been exposed to the emitted light for a defined time interval;
adjusting the flux output conditions based on the at least one response parameter;
obtaining a second measurement of at least one response parameter related to the algae solution after it has been exposed to emitted light having adjusted flux output conditions for a second defined time interval;
identifying an optimal set of flux output conditions from the at least one response parameter when the response parameter is determined to reach a threshold value;
outputting the optimal set of flux output conditions as an optimal temporal variation of flux output for the algae solution; and
controlling the at least one light source of the illumination apparatus in accordance with the optimal temporal variation of flux output.

In one embodiment, the carbon capture system includes a defouler. In another embodiment, the carbon capture system includes a carbon sensor. Preferably, CO₂ gas is supplied to the gas injection inlet of the carbonation device.

In another embodiment, the carbon capture system further includes at least one photovoltaic panel to provide power to the system. Preferably, the photovoltaic panel is installed at a location above the terraced illumination apparatus. More preferably, the photovoltaic panel is used to power the at least one light source of the illumination apparatus.

In a further embodiment, the carbon capture system includes an automated nutrient dosing system. The carbon capture preferably includes at least one bioinformation sensor. More preferably, the bioinformation sensor includes at least one of a fluorometer, DO sensor, pH sensor, and a CO₂ sensor. The automated nutrient dosing system preferably controls the supply of nutrients to the system based on measurements from the at least one bioinformation sensor.

Preferably, the carbon capture system includes a harvesting device, configured to harvest biomass from the carbon capture system. Preferably, the harvesting device is configured to perform polymer flocculation.

### BRIEF DESCRIPTION OF THE DRAWINGS

Preferred embodiments of the disclosure will now be described, by way of example only, with reference to the accompanying drawings in which:

FIG. 1 is an image of a side view of the terraced illumination apparatus of the carbon capture system according to one embodiment;

FIG. 2 is an image of a front view of the terraced illumination shown in FIG. 1;

FIG. 2A is an exploded view of one of the trays of the terraced illumination apparatus of FIG. 1;

FIG. 2B is an isometric view of a terraced illumination apparatus according to another embodiment;

FIG. 2C is a front view of the terraced illumination apparatus of FIG. 2B;

FIG. 3 is an image of the terraced illumination apparatus or the carbon capture system in use, according to one embodiment, showing algae solution flowing on the trays;

FIG. 4A is a diagram of the carbonation device of the carbon capture system according to one embodiment;

FIG. 4B is an annotated version of FIG. 4A showing the direction of fluid and gas flow within the carbonation device;

FIG. 5 is a diagram showing a configuration of a sensor for CO₂ dosing with concentrated CO₂ sources used in the carbonation device of the carbon capture system;

FIG. 6 is a diagram showing a configuration of a sensor for CO₂ dosing with air of flue gas used in the carbonation device of the carbon capture system; and

FIG. 7 is a diagram of an illumination surface according to an embodiment of the invention, showing light sources, a tray and a light-dispersing layer.

### DETAILED DESCRIPTION

### System Overview

The present invention provides a modular carbon capture system. It is modular, such that it may fit into a shipping container or an enclosed building space (such as a glasshouse or a basement). The system's terraced illumination surfaces enable multiple systems to be set up within a relatively small area, such as a shipping container, and can be transported and stored easily.

In one embodiment, there is provided a carbon capture system including an illumination apparatus. The illumination apparatus includes at least one tray, a supporting frame configured to receive the at least one tray, a light-dispersing layer disposed on the at least one tray, and at least one light source. The at least one tray includes a substantially planar base, with a peripheral rim extending around the base to form a receptacle for holding a fluid. The light-dispersing layer is disposed on a surface of the planar base to enable substantially uniform distribution of light through the planar base. The light source is positioned adjacent the planar base of the tray and configured to emit light toward the planar base such that at least a portion of the emitted light is dispersed by the light dispersing layer through the surface of the planar base.

The carbon capture system may further include a carbonation device. The carbonation device includes a fluid vessel, a fluid supply system disposed on an upper portion of the fluid vessel to supply fluid to the fluid vessel and a gas injection inlet disposed on a lower portion of the fluid vessel to supply gas to the fluid vessel. The device includes at least one mixing element arranged within the fluid vessel and below the fluid supply system. A diffuser is arranged within the fluid vessel such that gas supplied through the gas injection inlet passes at least partially through the diffuser to produce a diffused gas, and the diffused gas and fluid mix within the fluid vessel to produce a mixed fluid. An outlet is disposed on the fluid vessel to release the mixed fluid.

The carbon capture system may further include an assessment device in communication with the illumination apparatus. The assessment device is able to expose samples of microalgae or other photosynthetic organisms to a wide variety of combinations of frequency, intensity and duty cycle; with the specific intention of identifying the optimal conditions for max biomass production or max cellular component (such as pigment/cell or metabolite/cell). One embodiment of the assessment device is based on a 96-well plate with an array of LEDs controlled by a series of microchips driving the illumination of each well. Raspberry pi control units allow full manipulation of the frequency, intensity and duty cycle. Each test runs for at least 10 minutes of exposure followed by a fluorometric assessment of the impact via a CCD camera fitted with a chlorophyll a specific emission filter. The device consisting of the 96 LED array is modular and designed to couple together with multiple units for performing algae growth tests in hundreds of different flickering illumination conditions.

The carbon capture system may be configured to carry out a method for determining optimal temporal variations in flux output for algal growth. It will be appreciated that the term “flux” used herein may include luminous flux, radiant flux or both. The method includes the steps of providing an algae solution on the at least one tray, controlling that at least one light source to emit light towards the at least one tray in accordance with flux output conditions, wherein the flux output conditions include at least a duty cycle and a pulse frequency; exposing the at least one tray to the emitted light for a predefined time interval; obtaining a first measurement of at least one response parameter related to the algae solution after it has been exposed to the emitted light for a defined time interval; adjusting the flux output conditions based on the at least one response parameter; obtaining a second measurement of at least one response parameter related to the algae solution after it has been exposed to emitted light having adjusted flux output conditions for a second defined time interval; identifying an optimal set of flux output conditions from the at least one response parameter when the response parameter is determined to reach a threshold value; and outputting the optimal set of flux output conditions as an optimal temporal variation of flux output for the sample. In one embodiment, the carbon capture system controls the at least one light source of the terraced illumination apparatus to provide temporal variations in flux output. In some embodiments, the carbon capture system controls the at least one light source of the terraced illumination apparatus on the basis of identified optimal temporal variations in flux output.

In one embodiment, the carbon capture device is positioned within an enclosed space. The enclosed space may be a shipping container. The terraced illumination apparatus of the carbon capture system includes a plurality of terraced strays. The terraced trays form illumination surfaces upon which algae solution flows. The illumination surfaces are illuminated with an LED light source. The LED can be tuned with specific flux output conditions to minimise energy and maximise production using pulse width modulation of the frequency and duty cycle, as well as the optimum wavelengths delivered to enhance production. CO₂ delivery from the air into the liquid growth media is enhanced using the carbonation device of the carbon capture system. The system is designed to minimises off-gassing of CO₂. Where the carbon capture system is deployed in a shipping container, the shipping container can be air conditioned to maintain an optimal temperature. It can also containerise a PC2 facility allowing GM algae to be used.

Applications of the modular carbon capture system include biomass production close to sources of CO₂, nutrients or wastewater (for remediation). The carbon capture device also contributes to the circular bioeconomy. A unique species or consortia of algae may be selected (for example bred or screened) to suit the source and/or concentration of CO₂. For example, this may differ based on whether the CO₂ is flue gas or biogas). The algae may also be selected to suit the source of nutrients (for example, wastewater, anaerobic digester centrate, growth media). The growth irradiance may be optimised for each species. In some embodiments, this optimisation is identified using the assessment device which may be in communication with the carbon capture system. In other embodiments, this optimisation is identified using the method for determining optimal temporal variations in flux output. In other embodiments, the optimal temporal variations are identified using the assessment device and then applied to the terraced illumination apparatus of the carbon capture system.

Biomass from the carbon capture system can be converted into bioplastics, building materials and other permanent carbon products. Alternatively, the carbon capture system may also be used for other applications such as where the biomass is used for feed or food. The carbon capture system can be used in various industries including, but not limited to, wastewater/anaerobic digestion (AD) biogas production, carbon capture and manufacturing, construction abatement, fermented beverages, cement manufacturers, remote mining, CO₂ removal from building air conditioning, heavy industry with hard to abate CO₂ emissions, food waste AD facilities, and/or direct air capture.

### Terraced Illumination Apparatus

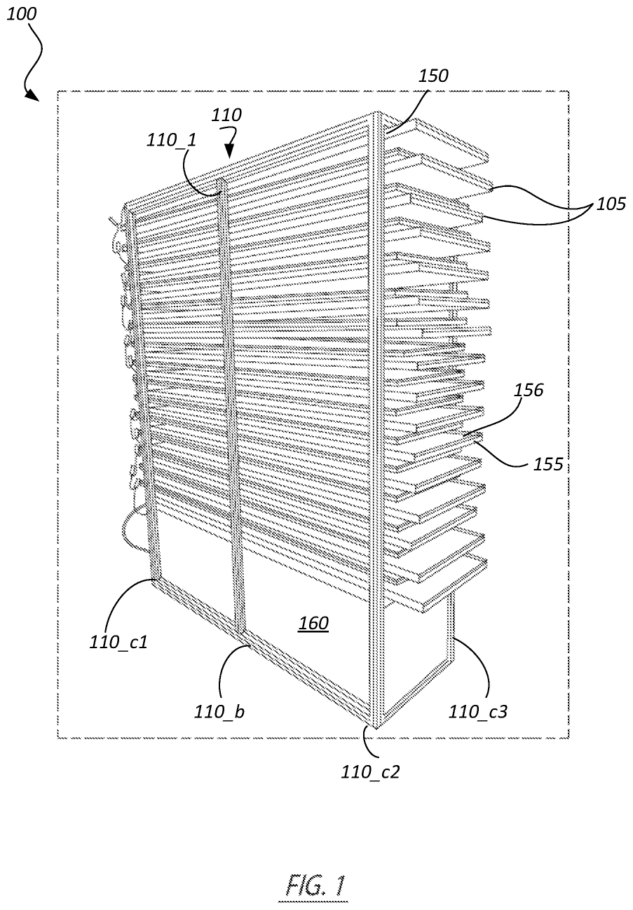

FIG. 1 shows an example 100 of the terraced illumination apparatus of the carbonation capture system according to a preferred embodiment of the invention. In the preferred embodiment, there is provided a plurality of trays 105, arranged vertically in a terraced arrangement, within a substantially rectilinear supporting frame 110. Each of the plurality of trays are received in the supporting frame by pairs of supporting flanges which extend inwardly from the supporting frame. The base of the trays at least partially abuttingly engage the supporting flanges to support the tray. Every second tray is offset against the tray above it such that each front or rear edge of the trays alternates between a proximate and a distal position.

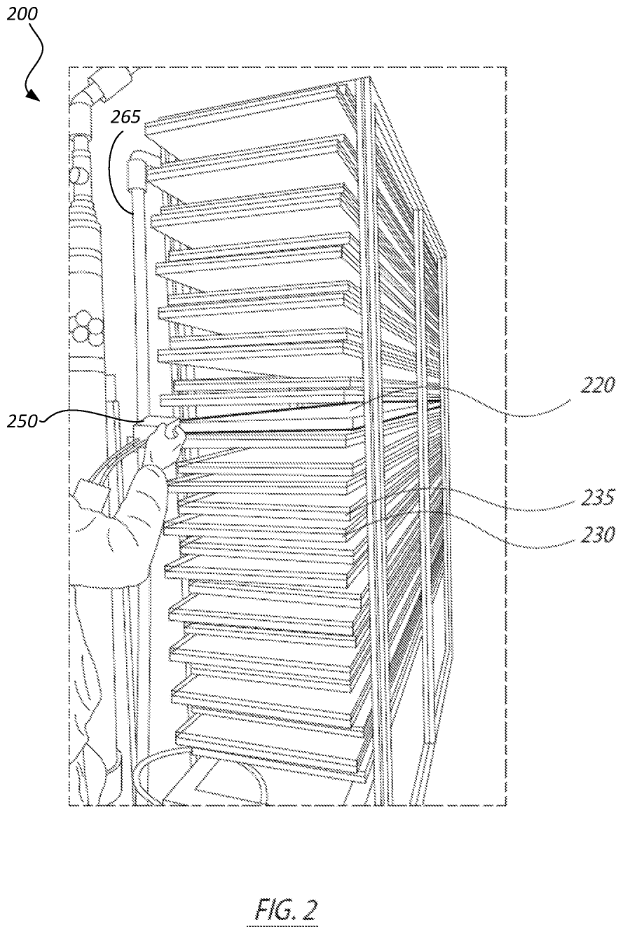

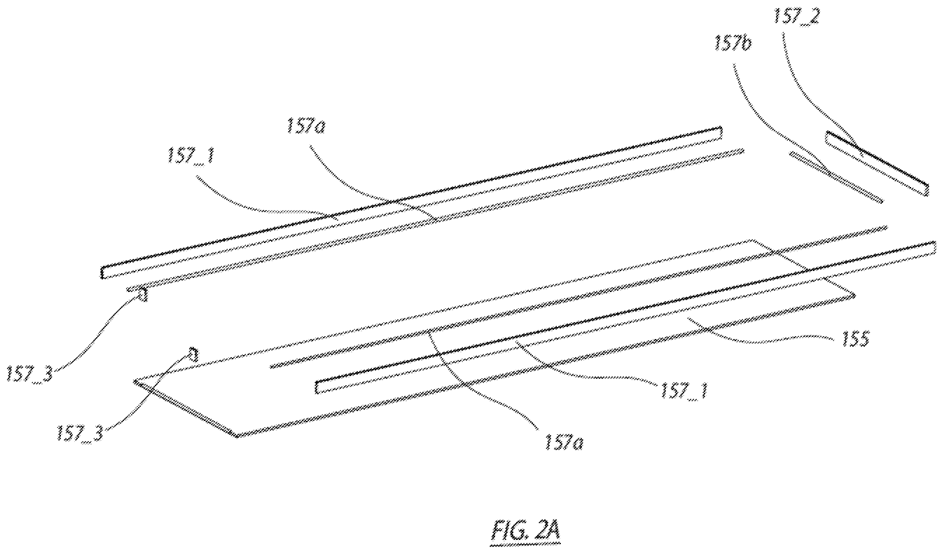

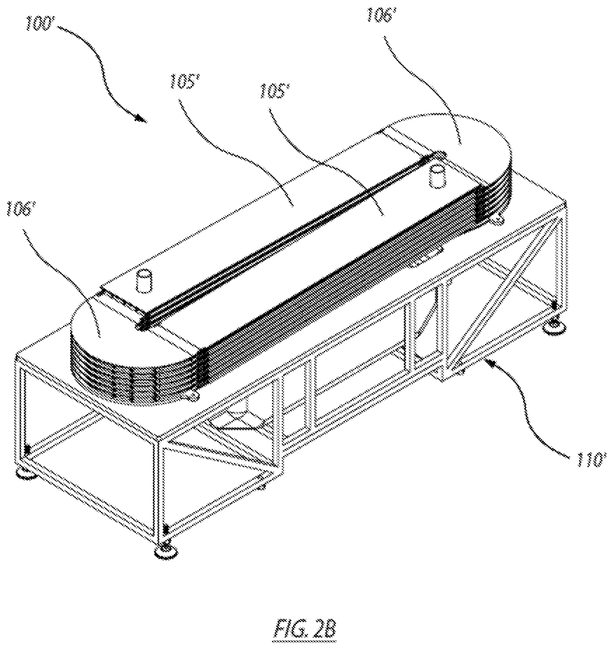

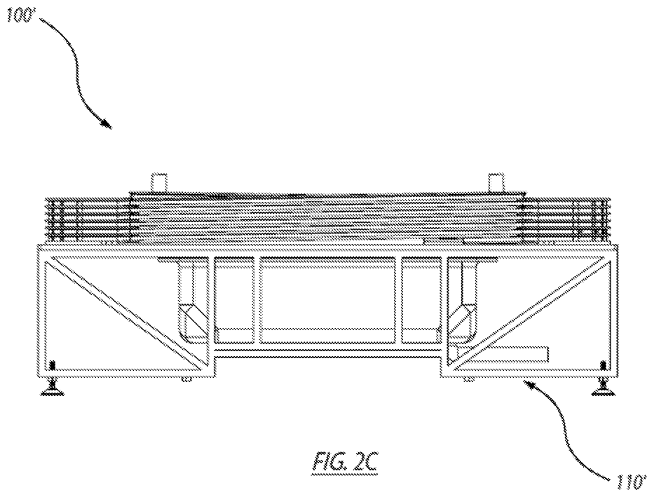

Advantageous aspects of illumination are demonstrated in an example 200 in FIG. 2, where the algae solution is distributed evenly on an illumination surface 210 formed from the light source (e.g., 250) being directed into the planar base of the tray (e.g. tray 220). Light energy is made immediately available to algae cells through the light-dispersing layer.

FIGS. 2B and 2C show an example 100′ of a terraced illumination apparatus according to another embodiment. The example 100′ includes a plurality of trays 105′ which are similar to the trays 105 of the example 100 apart from the trays 105′ being vertically aligned in a pair of oppositely sloped stacks. Each of the trays 105′ (apart from the uppermost and lowermost tray) is connected at respective ends thereof to a curved end portion 106′ of the apparatus so as to form a spiral fluid circuit arrangement. That is, the algae solution is circulated between the stacked layers of alternating trays 105′ and curved end portions 106′, which optimises the space utilisation of the apparatus of the example 100′. The apparatus may be sealed between each of the stacked layers of alternating trays 105′ and curved end portions 106′ and the trays 105′ covered to improve productivity. In the embodiment depicted, the apparatus of the example 100′ is supported by a frame 110′, but other embodiments are contemplated in which the apparatus of the example 100′ is configured to be self-supporting.

The terraced arrangement minimises footprint. For example, in one embodiment, the system achieves 30 m² of illumination surface over just 2 m² footprint and 2.5 m height space.

### Trays

With particular reference to FIG. 2A, the apparatus is provided with at least one tray. The tray may also be referred to as an illumination tray. The tray is in some embodiments substantially rectilinear and includes a substantially planar base, such as 155. In a preferred embodiment, the tray is substantially rectangular, having two symmetrical elongate side edges of equal length. The end edges are also symmetrical, being equal in width and shorter than the elongate side edges. In this case, the first end edge may be described as a front edge and the second end edge may be described as a rear edge. However, it will be appreciated that the terms front edge and rear edge are used for the purposes of description only, and do not limit the position of the tray. In some embodiments, the front edges of alternating trays may be located at opposite sides of the supporting frame, for example trays 230 and 235 of FIG. 2. The planar base of the tray is substantially flat. When an algae solution is dispersed across the tray, the base facilitates flow of the algal solution across the tray. In some embodiments, the algal solution is evenly dispersed across the tray. In other embodiments, the base may include a textured surface to slow the flow of algal solution, or to direct the algal solution along a predefined path.

The tray further includes a peripheral rim extending around the base 155 to form a receptacle for holding a fluid. In some embodiments, the rim comprises upright edges 157_1, 157_2 and 157_3. In some embodiments, the upright edges do not extend all the way along the perimeter of the tray, and may include breaks in the peripheral rim. In the embodiment depicted, reinforcement 157a, 157b is provided at the join between the upright edges 157_1, 157_2 and 157_3 and the base 155. The tray is configured to receive a flow of algal solution. The upright edges allow the tray to contain at least a 5 mm layer of algae solution. The upright edges are approximately between 10 mm and 50 mm in height and between 5 and 10 mm in thickness. Preferably, the upright edges are 30 mm in height and 6 mm in thickness. The upright edges extend from the planar base and are between approximately 500 mm and 2000 mm in length and 30 mm in height. The tray may be integrally moulded or formed. In other embodiments, the planar base may be formed separately to the peripheral rim, the rim being attached to the planar base after manufacture.

The tray is made from a transparent material to enable the light to pass through it. In one embodiment, the tray is made from an acrylic material. For example, the tray may be made from an acrylic sheet material, such as an acrylic Edge LED sheet, “Plexiglass” or “Acrylite”. In some embodiments, the acrylic sheet is made from methyl methacrylate monomer or polymethyl methacrylate. In other embodiments, the tray may be constructed from a suitable transparent material. For example, the tray may be made from glass or plastic.

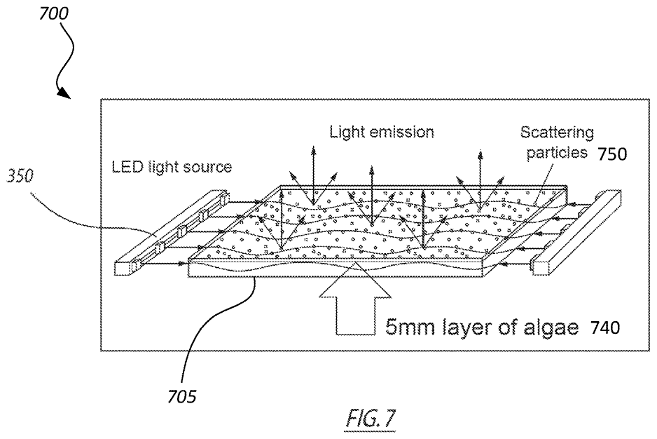

The tray includes a light-dispersing layer (e.g., 156 in FIG. 1) disposed on a surface of the planar base. Preferably, it is disposed on an upper surface. The light dispersing layer may be integrally formed with the planar base. Alternatively, the light dispersing layer may be a separate layer or material affixed on top of the planar base. The light dispersing layer should be at least partially transparent to allow the light to pass through it. The light-dispersing layer defines an illumination surface or a “glow surface” on the planar base of the tray, and essentially turns the trays into illumination surfaces. In one embodiment, the light-dispersing layer is an optical diffuser. The optical diffuser may include a transparent substrate with a textures or etched surface. The optical diffuser may be an optically diffusing film. As shown in FIG. 7, the optical diffuser may include a material embedded with diffusing particles or scattering particles. For example, the optical diffuser may include a diffractive plastic film. The diffractive plastic film may be constructed from polymeric material. In other embodiments, the light dispersing layer may be integrally formed with the tray, and the upper surface of the tray may be textured or etched with a diffuser pattern. The light-dispersing layer allows for uniform distribution of photon energy throughout the surface area of each tray.

FIG. 7 shows an example 700 of the light source 350, a tray 705 (corresponding to the try 105 for example), and a layer of algae 740. The embedded diffusing particles or scattering particles (for example 750) are adapted to evenly distribute light at desirable intensity to algae cells on the tray 705. As such, the light source 350 is in direct contact with algae solution 740 while minimizing light loss to space. In the arrangement of FIG. 7, the LED light source 350 can be arranged in a distance to minimize light loss due to absorption and dissipate heat. Illumination on a layer algae culture (5-7 mm) can also minimise light shading by algae solution. The terraced trays are arranged vertically to trap light within the photobioreactor.

In some embodiments, the peripheral rim includes at least one aperture to enable the algal solution to flow out of the tray. The aperture may be an elongate aperture that extends along the one edge of the peripheral rim of the tray to enable the algal solution to flow in a uniform manner out of the tray. The elongate aperture enables the algal solution to flow out of the tray at the same flow rate, or without substantially changing the flow rate. In other embodiments, the peripheral rim extends only partway around the tray. This may include one or more gaps in the peripheral rim as it extends around the planar base. In some embodiments, one edge of the tray is open. The rear edge may be the open edge of the tray, in which there is no peripheral rim. In some embodiments, the rear edge may be trimmed at a 45 degree angle in order to facilitate flow of the fluid over the rear edge. In a rectangular tray, the peripheral rim may extend around only three of the four edges of the tray, such that an open end is defined. For example, the rear edge may not include a peripheral edge, such that when an algal solution flows from the front edge towards the rear edge, the solution is able to flow freely over the rear edge and out of the tray. In other embodiments, the peripheral rim may include a shortened portion, in which at least part of the upright edge extends a shorter distance upwards than the remainder of the upright edges of the peripheral rim.

In a preferred embodiment, the apparatus includes a plurality of trays, as shown in the embodiments of FIG. 1 and FIG. 2. Each tray is received in the supporting frame such that they are vertically spaced apart in a terraced arrangement to provide a plurality of illumination surfaces. As shown in FIG. 1, the trays 105 are arranged one above the other with substantially equidistant spacing. The trays may be vertically aligned with each other. In some embodiments, the front edge of every second tray is offset against the front edge of every other tray by a predetermined amount, as shown in the arrangement of trays 230 and 235 of FIG. 2. This offset enables the algal solution to flow from each individual tray into the tray below it without spilling the algal solution. The trays may also be arranged at a predetermined angle within the supporting frame. In this embodiment, every second tray is arranged at an opposing predetermined angle, so as to form a Z-format arrangement of trays which facilitates the flow of algal solution from tray to tray. For example, a first tray (230) may be disposed such that the rear edge of the first tray is lower than the front edge, and therefore the first tray slopes downwards towards the rear edge. In a second tray (235), located underneath the first tray, the rear edge of the second tray is higher than the front edge, and therefore the second tray slops downwards towards the front edge. The trays alternate in this patent to form a ‘Z’ pattern when viewed from the side.

The slope of the trays may be varied from 0.5% to 1.5% gradient. Preferably, the slope of the trays is 1%. The slope selection in the tray arrangement determines the number of trays per meter of height and the algae solution flow rate. When the terraced tray system is initially set up, the slope, and therefore the number of trays may be determined based on maintaining the most illumination surfaces per square meters. That is, the flow rate of the algal solution on the trays can be controlled by changing the slope of the trays. The supporting flanges of the supporting frame may be arranged on a predetermined angle to enable each tray to sit at the same predetermined angle. In other embodiments, the supporting flanges may be adjustable to select a slope for each of the terraced illumination trays. The arrangement of the plurality of trays is such that the algal solution is provided at the front edge of the topmost tray and is able to flow across the planar base of the topmost tray towards the rear edge and into the tray located below.

### Light Sources

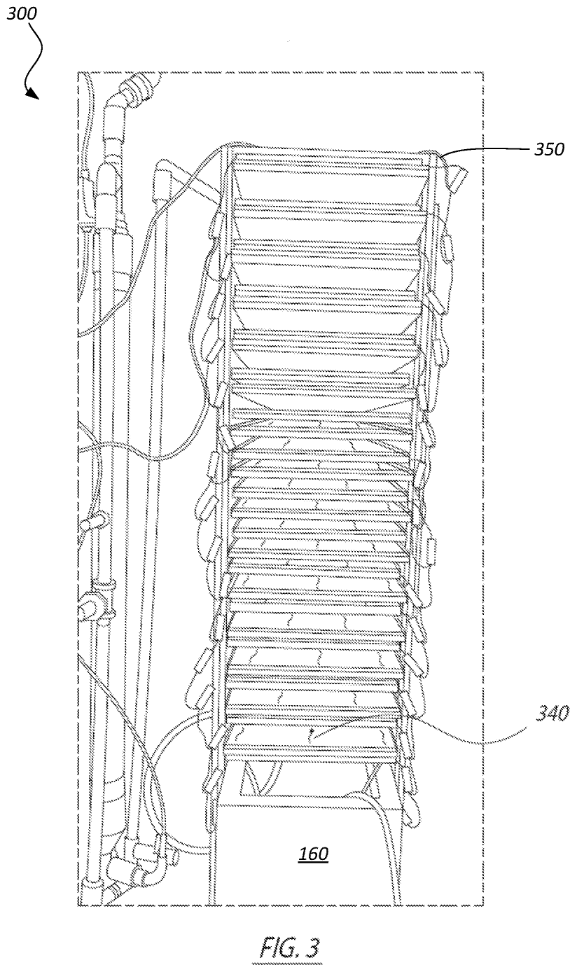

The apparatus is also provided with at least one light source (for example 350 in FIG. 3). The light source is positioned adjacent to the planar base of the tray and configured to emit light toward the planar base such that at least a portion of the emitted light is dispersed by the light-dispersing layer through the surface of the planar base. Light waves are channeled horizontally through the planar base of the tray and then diffracted vertically by a light-dispersing layer to form an illumination surface. This diffracts the light directly to a thin layer of algae solution provided by the trays. Within the apparatus, where a plurality of trays are used, the light sources create multiple illumination surfaces to cultivate the algae solution, thereby achieving a ratio of cultivation surface over volume which is substantially higher (up to 100 times more) than the traditional raceway system.

The light source may include a light-emitting element (LEE). A light emitting element may be any device that emits electromagnetic radiation at a defined wavelength. For example, visible, infrared or ultraviolet wavelengths. The emitted light is preferably in the visible spectrum, but may also include infrared or ultraviolet wavelengths. The light emitting elements may be activated by passing a current through the element, or applying a potential difference across the element. The light-emitting elements may include a semiconductor device. The light-emitting element may include, but is not limited to, solid-state, organic, polymer, phosphor-coated or high-flux LEDs, and/or laser diodes. The light source is a light-emitting element including at least one light emitting diode (LED). In some embodiments, the LED is a cool white LED.

The light source may include an array of light-emitting elements. In a preferred embodiment, the light source is a light-emitting element in the form of a plurality of LEDs. The plurality of LEDs are arranged in an array and to provide light to the planar base. In some embodiments, the plurality of LEDs are mounted directly on the front edge and rear edges of the tray. In other embodiments, the LEDs are mounted on at least one of a side edge, front edge and rear edge of the tray. In an alternative embodiment, the plurality of LEDs are mounted on at least one of the pair of supporting flanges of the supporting frame, such that the light is emitted towards the planar base. The array of LED's may be arranged on an elongate bar attachable to the supporting frame. In a further embodiment the array of LED's may be arranged on a mount attachable to the at least one tray. The LEDs may be arranged on an adhesive strip which is applied to the supporting flange. Alternatively, the LEDs may be arranged on an upright edge of the supporting flange. For example, the LEDs may be installed on a vertical or horizontal inner surface of an L-shaped bar. Similarly, they may be installed on a vertical or horizontal inner surface of a U-shaped bar. The LEDs may alternatively be mounted at least partially on the support frame. In yet a further embodiment, the array of LEDs are arranged or mounted on an elongate bar, configured to slidingly engage with at least one of the at least one pair of supporting flanges. By mounting the LED's on a separate elongate bar, this enables easy replacement and troubleshooting for the light source in the apparatus. The elongate bar may be in the form of an aluminium angle. Preferably, the plurality of LEDs are mounted on an aluminium angle in the same shape as the supporting flange. In another embodiment, the array of LEDs is attachable to at least one supporting flange, and the at least one supporting flange is formed from an aluminium angle attachable to the supporting frame.

The plurality of LEDs may be mounted on the apparatus by using an adhesive, an adhesive intermediate material, a separate mount which is attachable to the tray of supporting frame, or any other suitable means of mounting the light source. In some cases, the light sources are applied to every second tray, such that the apparatus provides an arrangement of vertically stacked terraced illumination trays in which every second tray has a light source emitting towards the planar base, and the remaining trays do not have a direct light source emitting towards their base, by use the light from the trays above and below them.

### Frame

The apparatus includes a supporting frame (such as a frame 110) that is configured to receive at least one tray. The supporting frame is generally rectangular. Preferably the supporting frame 100 is configured to receive a plurality of trays. The supporting frame 110 extends upwards from a substantially rectangular base 110b. The supporting frame 110 is substantially rectilinear in shape. However, it will be appreciated that the shape of the support frame may take other shapes. For example, in one embodiment, the frame may be substantially cylindrical. The frame 110 includes at least two support shafts (such as 110_1) which extend upwards from the base. Preferably, the frame 110 includes four corner shafts which extend upwards from each the four corners of the base and form the main body of the supporting frame (for example 110_c1, 110_c2 and 110_c3). The frame may further include at least two support shafts which each extend upwards from each one of the side edges of the rectangular base. These support shafts provide additional support to the main body of the frame, and are used to mount the supporting flanges (e.g., 150) used to receive the trays. A plurality of support shafts may be provided on each side edge to provide additional support, and more support shafts will be needed the longer the rectangular base becomes, and the greater the distance between the four corner shafts.

The supporting frame 110 also includes at least one pair of supporting flanges 150 disposed symmetrically on opposing sides of the frame 110. The supporting flanges 150 are disposed on the main body of the supporting frame and extend inwards. Preferably, the supporting flanges 150 are attached to at least two corner shafts and a support shaft. Each pair of flanges 150 is symmetrically disposed on the inside edges of opposing sides of the main body of the supporting frame. The supporting frame 110 may include a plurality of pairs of supporting flanges 150 for receiving a plurality of trays 105. In some embodiments, the flanges are disposed perpendicular to the corner shafts and the support shafts. In further embodiments, the plurality of pairs of flanges are spaced equidistantly in parallel to each other, so as to allow the received trays to be equidistantly spaced. Preferably, the supporting flanges 150 provide a surface upon which at least part of the planar base 155 of the tray abuttingly engages with the pair of supporting flanges 150 to be supported within the frame 110. The pair of supporting flanges may be disposed at predetermined angle on the support frame in order to allow the trays to be positioned at a predetermined angle. For example, the supporting flanges may be arranged, in pairs, to form a ‘Z’ pattern or a Z-format arrangement, to enable the trays to slope upwards or downwards to control the flow of the algal solution.

The supporting flanges may be in the form or an L-shaped or U-shaped elongate bar. In other embodiments, the supporting flanges may by upturned flanges, or a V-shaped bar, configured to engage with a corresponding downturned flange extending from the tray. In further embodiments, the tray may include an outwardly extending flange to abut the pair of supporting flanges. In yet further embodiments, the supporting flanges may be configured to slidably engage with the trays to enable the supporting frame to receive the trays. In an alternative embodiment, the supporting frame may include a plurality of pairs of symmetrically disposed recesses, instead of supporting flanges, within which a plurality of trays could be received by the supporting frame. In another embodiment, the supporting flanges may form a track that may be operatively engaged by roller wheels attached to the planar base of the tray.

In further embodiments, the base of the supporting frame includes a tank 160. The tank 160 may also be received within the base of the supporting frame, such that the tank 160 is removable and/or replaceable. The main body of the support frame 110, and/or the base of the support frame 110, may be configured to fit around the tank 160. In other embodiments, the tank 160 may be integrally formed with the supporting frame 110. The tank 160 is a fluid holding tank. In a preferred embodiment, the tank 160 holds algae solution for dispersion onto the trays. The tank 160 may include an outlet from which extends a pipe (for example 265 of FIG. 2) that carries the algal solution to at least one tray, such that fluid travels between the tank 160 and the at least one tray. In some embodiments, the algal solution is pumped vertically up the pipe 265 to the topmost tray. The tank may further include an inlet for receiving algal solution. Alternatively, or additionally, the tank may have an open top, or include at least one aperture to receive algal solution. The tank may receive algal solution from the plurality of trays, from a secondary tank and/or from a carbonation unit. The tank and the plurality of trays form a fluid circuit. That is, the algae solution is circulated to the first tray, flows down the trays disposed beneath the first tray, and eventually returns to the tank 160 by gravity. As the tank 160 is positioned below the plurality of trays 105 within the supporting frame 110, this optimises the space utilisation of the apparatus.

In one embodiment, the tank 160 is filled with an algal solution. The algal solution is pumped from the tank 160, through a pipe (for example 265) and up to the top tray in the terraced illumination apparatus. The algal solution is then supplied to an upper end of a topmost first tray such it flows towards a lower end of the tray. Once it reaches the lower end of the first tray, it flows onto an upper end of a second tray. The second tray is the tray beneath the first tray. The algal solution then flows from the upper end of the second tray to the lower end of the second tray, and then over onto an upper end of a third tray which is located beneath the second tray. The algal solution continues to flow from an upper end of a tray to a lower end of a tray and then onto the tray located directly beneath it until the algal solution has flowed across each individually tray in the terraced illumination apparatus. Once it flows over the lower end of the lowest final tray, the algal solution then flows into the holding tank 160 in the base of the support frame. FIG. 3 shows one embodiment 300 of the carbon capture system with the terraced illumination apparatus in use having algae solution flowing on each of the trays, for example marked as 340 on a lower tray.

In other embodiments, the algae solution may be dispersed on the trays directly from a fluid vessel of a carbonation device.

### Carbonation Device

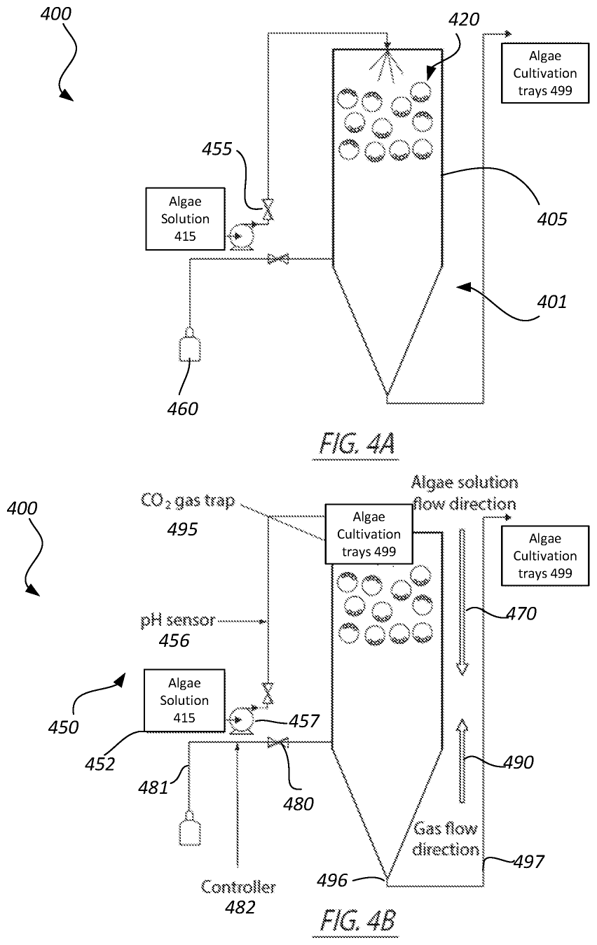

The carbonation device is able to carbonate a microalgae culture effectively and efficiently, and different scales. An example carbonation device 400 is shown in FIGS. 4A and 4B. The carbonation device 400 may also be referred to as a carbonation jet. The carbonation device 400 facilitates the interface of CO₂ gas bubbles with a microalgae solution in an enclosed column 405 from which highly carbonated algae solution will flow out for algae photosynthesis. In the carbonation device 400, the CO₂ gas and algae solution flow in opposite directions. CO₂ gas is introduced via air stone 460 at the lower portion of the column 405. Algae solution 415 is pumped in on the top of the column 405. CO₂ gas bubbles upwards and hits the water flow. The column 405 includes a plurality of floatable balls 420 as a mixing element disposed at the CO₂ gas and algae solution injection points. These floatable balls 420 create static mixing and shear of CO₂ gas and algae solution on their surface. The saturated CO₂ algae solution flows out at the bottom of the column to distribute onto terraced illumination layers for algae cultivation.

The dissolved CO₂ in the algae solution is in three different species (CO₂, HCO³⁻ and CO₃²⁻) with concentrations depending on the pH level of the algae solution. Amongst these species, algae can use most of CO₂ and HCO³⁻. The relevant dose of CO₂ to the carbonation jet is controlled on the basis of the detected pH level of the algae solution. The carbonation device 400 provides substantially no CO₂ gas loss, high CO₂ transfer, and easy control to match CO₂ demand by the algae. CO₂ dissociation into a fluid depends on pH and retention time. The device of the present invention provides both conditions for efficient mass transfer.

In one embodiment, fluid vessel 401 is enclosed. By enclosing the column 405, this prevents the gas from escaping at a surface of the fluid. In other embodiments, the fluid vessel 401 may be open. For example, the top of the fluid vessel 401 may be open at the top, or may include an aperture which extends at least partially across the top of the fluid vessel. The fluid vessel 401 may be column-shaped and elongate, such that it has an upper portion and a lower portion. In some embodiments, the lower portion is defined by the lower two-thirds of the fluid vessel 401, and the upper portion is defined by the upper one-third of the fluid vessel 401. The fluid vessel 401 may be substantially cylindrical in shape. However, it will be appreciated that the fluid vessel 401 may be in other forms such as rectilinear or prismatic configurations. The fluid vessel 401 should provide enough area for the fluid and gas to adequately mix. The more contact that the gas has with the fluid within the fluid vessel, the greater the amount of gas dissociation within the fluid. The fluid vessel 401 may have a volume of between 5 L and 20 L. However, it will be appreciated that the size and volume of the vessel can be changed depending on the scale of the algae solution. In one embodiment, the fluid vessel is designed for a 2.5 m³ algae culture. In a further embodiment, the vessel 401 is 12 L, with a height of about 1500 mm and diameter: of about 100 mm.

A fluid supply system 450 is disposed on an upper portion of the fluid vessel 401 to supply fluid to the fluid vessel. In some embodiments, the fluid supply system 450 is located on the upper one-third of the fluid vessel. The fluid supply system may be disposed on the top of the fluid vessel. Alternatively, the fluid supply system 450 may be located on a side of the fluid vessel 401. In one embodiment, the fluid supply system 450 includes a fluid supply inlet 455 through which fluid is supplied to the vessel. The fluid supply inlet 455 may be located on the top of the fluid vessel 401, or it may be located on a side of the vessel. In some embodiments, the fluid supply system 450 may include a plurality of fluid supply inlets. The fluid supply system 450 may also include a supply pipe through which fluid is transported to the fluid supply inlet. The supply pipe enables the fluid supply system to be in fluid communication with at least one storage tank (e.g., 452), such that fluid flows from the at least one storage tank to the fluid supply system 450. The at least one storage tank 452 may include a pump 457 to enable the fluid 415 to be pumped to the fluid supply system 450 at the top of the fluid vessel 401. At least one sensor may be positioned within the supply pipe. The at least one sensor may be a pH sensor (e.g., 456). In one embodiment, there may be a plurality of sensors. The carbonation device 400 may further include a dissolved CO₂ sensor. In some embodiments, a pH sensor and a dissolved CO₂ sensor can be used to measure the fluid flow out of the fluid vessel. In further embodiments, the carbonation device may include an atmospheric CO₂ sensor for measuring concentration of CO₂ in the atmosphere. The pH sensor 456 is configured to measure the pH of the fluid before it enters the fluid vessel. The fluid supply inlet 455 includes a valve configured to regulate inflow of the fluid. The valve may include a check valve to prevent backflow of the fluid from the fluid vessel back through the supply inlet. The fluid supply system 450 may also include at least one supply sensor. The supply sensor may include a volume meter for measuring the amount of fluid flowing into the fluid vessel.

The fluid supply system 450 may include at least one spray nozzle for introducing the fluid to the fluid vessel. In an alternative embodiment, the fluid supply system 450 may further include a manifold adjacent to the fluid inlet such that the fluid passes through the manifold and into the fluid vessel. The manifold may be attached to the fluid inlet. The manifold may include at least one outlet through which the fluid leaves. The manifold may include a plurality of outlets through which the fluid leaves the manifold and enters the fluid vessel. The plurality of outlets may include a plurality of spray nozzles. The spray nozzles may provide an increased fluid velocity with which the fluid enters the vessel. The fluid 415 is supplied to the fluid vessel 401 such that the flow direction of the fluid is downwards with respect of the fluid vessel as indicated by arrow 470. That is, the direction of fluid flow is towards the lower portion of the vessel. In one embodiment, the fluid provided by the fluid supply system is a liquid. The liquid may be an algae solution. The algae solution may be an alkaline algae solution. An alkaline algae solution may provide better dissociation than water. By using an alkaline solution, dissolved CO₂ turns into HCO³⁻ which is the ideal form of carbon for algae to use. Furthermore, in the form of HCO³⁻, it is possible to prevent the release of CO₂ back to the atmosphere. Additionally, utilising an alkaline algae solution enables the injection of CO₂ in a precise manner so as not to kill algae.

A gas injection inlet 480 is disposed on a lower portion of the fluid vessel 401 to supply gas to the fluid vessel. In some embodiments, the gas injection inlet 480 is provided on the lower third of the fluid vessel 401. The gas injection inlet 480 may be disposed on a side of the fluid vessel. The gas injection inlet 480 may include a valve to regulate injection of the gas into the fluid vessel 401. The gas injection inlet 480 may be connected to a gas pipe 481 which supplies gas to the gas injection inlet. The gas pipe 481 may include a controller 482 to control the amount of gas being injected into the fluid vessel at any time. The controller 482 may be in the form of a switch or valve. The controller 482 may control the gas being supplied through the gas injection inlet 480 on the basis of at least one characteristic of the fluid. The at least one characteristic of the fluid may be a pH level. In some embodiments, the controller 482 may be in communication with the at least one sensor 456, configured to obtain measurements of characteristics of the fluid to then adjust the dosing of the gas accordingly.

The gas can be injected directly into the enclosed vessel 401, meaning that there is substantially no gas loss. The gas may be dosed upon algae consumption, which increases the efficiency and ease with which the carbonation device can be scaled up. That is, where the fluid vessel is larger in volume, more gas can be injection as required.

In another embodiment, the gas supply inlet 480 may extend through the side of the fluid vessel 401 and into the fluid vessel 401 such that the gas is injected centrally with respect to the fluid vessel. In other embodiments, the gas supply inlet 480 is configured to inject gas through a diffuser.

A diffuser may be arranged within the fluid vessel 401 such that gas supplied through the gas injection inlet 480 passes at least partially through the diffuser to produce a diffused gas. The gas may be diffused into a plurality of microbubbles to define the diffused gas. In some embodiments, the gas injection inlet 480 is attached to the diffuser. In other embodiments, the gas injection inlet 480 injects gas into an intermediate pipe or an intermediate area before reaching the diffuser. The gas injection inlet may inject gas in an area below the air stone 460. The diffuser may be in the form of a diffusion plate. Alternatively, the diffuser may be in the form of an air stone. The air stone may be substantially round. The air stone may be substantially spherical or disc-shaped. Alternatively, the air stone may be cylindrical in shape. In some embodiments, the air stone is the same diameter as the fluid vessel 401. The air stone may be constructed from at least one of wood, stone, fibreglass, limewood, or glass. In one embodiment, the air stone is constructed from limewood. The air stone may have a pore size of between about 3 microns and 4 microns. The air stone may be cylindrically shaped.

When the gas is injected into the vessel 401, the gas passes through the diffuser and bubbles upwards with respect to the fluid vessel 401. The gas is supplied to the fluid vessel 401 such that the flow direction of the gas is upwards with respect of the fluid vessel, as shown by an arrow 490. That is, the direction of gas flow is towards the upper portion of the vessel 401. Once injected into the fluid vessel, the gas contacts the fluid in an opposite flow direction, as indicated in FIG. 3B. Shear and brush between the fluid and the gas enhances the gas dissociation. This provides significant contact time between the fluid and the gas to improve the amount of gas supplied to the fluid. The diffused gas and the fluid mix within the fluid vessel 401 to produce a mixed fluid.

In one embodiment, the gas supplied through the gas injection inlet is carbon dioxide (CO₂). In embodiments where the fluid 415 is an algae solution, the CO₂ is dissolved in the algae solution to provide a mixed fluid in the form of a carbon saturated algae solution. The diffused CO₂ in the algae solution may be in three different species: CO₂, HCO³⁻ and CO₃²⁻. The concentrations of each depending on the pH level of the algae solution. Algae can use most of CO₂ and HCO³⁻. The amount of CO₂ provided by the gas injection inlet is controlled by the pH of the algae solution. In this way, the carbonation device 400 is able to easily control and match the specific CO₂ demand required by the algae. In other embodiments, carbonation device 400 may be in communication with a carbon capture device such that the CO₂ gas is supplied from atmosphere or flue gas, or from a concentrated source.

The device further includes at least one mixing element (420). The mixing element 420 is arranged within the fluid vessel and below the fluid supply system. In some embodiments, The mixing element 420 is arranged within the upper portion of the fluid vessel 401. Alternatively or additionally, the mixing element 420 is arranged in a lower portion. The at least one mixing element 420 defines a gas trap 495 within the fluid vessel. As the gas is injected into the vessel and bubbles up through the diffuser, the mixing element 420 acts as an obstacle on which a concentration of the gas bubbles becomes trapped. The gas may be trapped on the surface areas of the mixing element 420. Alternatively or additionally, the gas bubbles may become trapped within crevices or gaps in the mixing element 420. In some embodiments, the mixing element 420 may be a static mixer. The static mixer may include a helical static mixer or a plate type static mixer. In other embodiments, the static mixer may include a spherical static mixer. The at least one mixing element may be attached to the inner sides of the fluid vessel. The mixing element may be rotatably mounted to the inside of the fluid vessel.

In some embodiments, there may be a plurality of mixing elements 420. The mixing elements 420 may be substantially round, or substantially spherical. The mixing elements 420 may be at least partially buoyant. When the mixing element 420 is partially buoyant, it does not need to be attached to the inside of the fluid vessel, but may sit in the upper portion of the fluid vessel by nature of its buoyancy. In other embodiments, the buoyant mixing element may be attached or partially attached to fix it in a specific area (such as in the lower portion of the fluid vessel) or within a predefined area (such as keeping it within in a range of the upper portion of the vessel). In some embodiments, a barrier is used to hold the buoyant mixing element in place, such as a grate placed within the fluid vessel that allows the fluid and gas to pass through but prevents the buoyant mixing element from rising above a predetermined height. In one embodiment, the at least one mixing element is a floatable ball. The mixing element provides static mixing of the fluid and the gas. In one embodiment, the device includes between 1 and 20 floatable balls as the mixing element 420. The floatable balls create static mixing and shear of the gas and fluid on their surfaces.

The floatable balls 420 create a large amount of surface area to trap the gas and increase the contact time between the gas and fluid to enable more shear and brush and increase gas dissociation within the fluid. The floatable balls 420 can easily be scaled to different sized systems and controlled by increasing the number of mixing elements in the vessel.

An outlet 496 is disposed on the fluid vessel 401 to release the mixed fluid from the fluid vessel 401. In some embodiments, the outlet 496 is disposed on the base of the fluid vessel 401. In other embodiments, the outlet 496 is disposed on a side of the fluid vessel 401. The outlet 496 may be positioned below the diffuser. This enables the mixed fluid to be released from the fluid vessel 401 without substantive gas loss. The outlet 496 may include at least one valve. The at least one valve may be at least one of a backflow preventer valve, a check valve, non-turn valve, reflux valve, retention valve, foot valve or one-way valve. The outlet 496 may be connected to an outflow pipe 497 to transport the mixed fluid away from the fluid vessel. In some embodiments, the outflow pipe 497 is also connected to a secondary vessel. The secondary vessel may be an algae solution holding tank. Alternatively, the outflow pipe may transport the mixed fluid to a cultivation unit 499. The cultivation unit 499 may be a photobioreactor. Alternatively, the cultivation unit 499 may be a terraced illumination apparatus (similar to 100 of FIG. 1). The outflow pipe 497 may transport the mixed fluid to at least one of a plurality of cultivation trays. The cultivation trays may be illuminated surfaces.

In one embodiment, an algae solution 415 is supplied by the fluid supply system 450 to the fluid vessel 401. The fluid vessel 401 is a cylindrical column. The algae solution 415 is pumped from a separate tank through the fluid supply inlet 455 provided on the top of the fluid vessel 401. The algae solution 415 flows downwards towards a lower portion of the fluid vessel 401. At the same time, CO₂ is injected into the fluid vessel through the gas injection inlet 480 at the lower portion of the column. The CO₂ gas is provided to the column on the basis of measurements obtained by a pH sensor (456) positioned on a supply pipe (e.g., 457) that feeds into the fluid supply inlet 455. The pH sensor determines the pH level of the algae solution, and a controller (182) then controls the amount of CO₂ dosing required for the algae solution having a particular pH level. In some embodiments, the pH of the algae solution is maintained above 8.5 to minimize dissolved CO₂ level.

The CO₂ gas passes through the diffuser as it enters the fluid vessel 401. The diffuser may be in the form of an air stone which is the same diameter as the cylindrical column, ensuring that the gas must pass through the diffuser. The air stone is positioned proximate the gas injection inlet such that the CO₂ gas is diffused into microbubbles as it hits the fluid flow. The CO₂ gas bubbles move upwards with a flow direction towards the upper portion of the column 405. The algae solution 415 flow direction is opposite to the gas flow direction. As the gas bubbles move upwards, the gas bubbles hit the mixing element 420. In this embodiment, the mixing elements are in the form of 20 floatable balls, disposed within the fluid vessel. Given their buoyancy, they sit at the top of the fluid vessel just in front of the fluid supply inlet. However, there is also one floatable ball provided just above the diffuser such that the CO₂ hits this almost immediately when entering the column. The floatable balls provide a large surface area for bubbles to be trapped, and the plurality of floatable balls therefor defines a gas trap. Substantially no CO₂ is lost to the atmosphere when using the device.

As the fluid moves past the gas trapped on the surface 495 of the floatable balls, enhanced shear and brush occurs to mix the CO₂ and the algae solution, and disperse the CO₂ into the algae solution. This then provides a saturated CO₂ algae solution. The saturated CO₂ algae solution flows out the outlet positioned at the bottom of the column. The saturated CO₂ algae solution can then be provided, e.g., to a photobioreactor. Specifically, the carbonated algae solution can be provided to a plurality of terraced trays or illuminated surfaces to cultivate algae.

### Assessment Device for Optimised Temporal Variations in Flux Output

The carbon capture system may additionally include a separate assessment device in communication with the carbon capture system for determining optimal temporal variations in flux output for algal growth. Flux output may also be referred to herein as “illumination”. Temporal variations in illumination can have a large effect on growth of algae. In a preferred embodiment, the assessment device includes at least one secondary light source, a sample plate affixed above the at least one secondary light source, a processor configured to control the secondary light source to provide a flux output, and at least one sensor.

The assessment device includes a sample plate configured to receive or contain a sample. The sample plate may be a microplate. The microplate is affixed above the at least one light source, configured to receive at least one sample. In some embodiments, the microplate is a single-well microplate. The single-well microplate may be in the form of a tray or receptacle for holding samples. In further embodiments, the microplate includes a plurality of wells. The plurality of wells may be arranged in an array, and may be equally spaced apart from each other. The microplate may be a 96 well microplate. The 96 well microplate is arranged in a 12 by 8 formation, with the center of each of the wells being spaced about 9 mm apart. The 96-well microplate is arranged to sit directly above an array of 96 LEDs, with each LED corresponding to a single well. The wells of the microplate may be between about 2 mm and about 15 mm in depth, and particularly between about 6 mm and about 13 mm, and more particularly between about 10 mm and about 12 mm. The wells of the microplate may be between about 4 mm and about 10 mm in diameter, and particularly between about 6 mm and about 8 mm in diameter. Preferably, the wells of the microplate are approximately about 4.2 mm in depth and about 2.7 mm in diameter, and holding a culture volume of about 200 μL. The wells of the microplate may have a U-shaped, V-shaped, F-shaped, or C-shaped base.

Preferably, the sample provided in the wells of the microplate includes algae. The algae may be a microalgae. Alternatively, the algae may be a cyanobacteria. Alternatively, the sample may include other photosynthetic material. While the embodiments herein reference algae, it will be appreciated that the method and system can be applied to various plants, plant materials, bacteria and archaea, and that the devices, systems and methods can be used in various applications including, but not limited to, horticultural, agricultural, and aquacultural environments, as well as commercial glasshouses, hydroponics, tank-based seaweed, and vertical farming production.

The assessment device includes at least one secondary light source able to emit light in accordance with temporally varying flux output conditions. That is, the secondary light source is able to provide illumination to a sample with a specific “flickering” pattern.

In a preferred embodiment, the secondary light source is an array of light-emitting elements, where the light-emitting elements are a plurality of LEDs. The LEDs may be arranged in a rectangular array. Preferably the LEDs are equidistantly spaced apart from each other. The LEDs may be arranged in a 12 by 8 grid, so as to align with individual wells (such as 720) of a 96 well sample plate. However, it will be appreciated that the number of LEDs does not need to align with the number of wells on the microplate. In some embodiments, the number of LEDs provided is the same as the number of wells of the microplate. In other embodiments, the number of LEDs per well of a microplate may be more than one.

The LEDs are in communication with a processor, which is configured to control the LEDs to provide illumination. The illumination or flux output provided by the LEDs includes at least a duty cycle and a pulse frequency. In further embodiments, the flux output also includes an intensity. The processor may include a controller, which is connected via electrical conductors to the LEDs. The controller controls the LEDs to provide illumination in accordance with particular flux output conditions. The processor controls a drive current to be delivered to the LED, and may be controlled by analog circuity. In an alternative embodiment, the drive current may be controlled by digital circuitry, and involve digitally modulating a signal to the LED.

The processor is able to set the flux output conditions, including a duty cycle, pulse frequency and intensity, of the emitted light, and provide temporally modulated drive current to the light source on the basis of these flux output conditions. The light source then emits light towards at least one sample in the microplate with illumination corresponding to those flux conditions. In some embodiments, the processor is configured to receive a signal in the form of a measurement obtained by at least one sensor. The processor can then incorporate the obtained measurement and adjust the flux output, thereby creating a closed loop feedback system for self-adjusting the temporal variations in illumination in real time to provide optimised conditions. Additionally, the ability for the processor to control the drive current to the LEDs enables more efficient energy usage by avoiding continuous current and utilising intermittent current.

The assessment device further includes at least one sensor. The sensor is configured to obtain a measurement of at least one response parameter of the at least one sample. The sensor may include a measurement sensor for measuring physical growth of the sample, a temperature sensor, an infrared sensor, gas sensor, photorespiration sensor, near-infrared sensor, pH sensor, colorometric sensor, or emission sensor. For algae cultivation, the at least one sensor monitors one or more characteristics associated with the algae sample. For example, to determine the growth rate of the algae sample and determine whether the growth rate is increasing or stagnating under the provided illumination conditions.

The sensor is preferably a CCD camera. The CCD camera may be fitted with a chlorophyll a specific emission filter. The emission filter enables a fluorometric assessment to be performed to ascertain the impact of the illumination conditions on the sample. In other embodiments, the camera may be fitted with other appropriate optical absorption or emission filters.

The response parameter may include any parameters or characteristics relating to the sample growth which are then used to optimise the provision of the temporal variations in illumination. For example, the chlorophyll a fluorescence may be monitored with at least one sensor and form part of the response parameter to determine whether the illumination conditions are impacting the sample growth. That is, the response parameter may provide a measure of how the sample is responding to the illumination conditions. In another embodiment, the response parameter may include the optical density of the sample. The optical density of the sample measured between 670 nm and 750 nm can be used to determine whether the algae is growing better under particular flux output conditions. In another embodiment, a plate reader may be used with a dye to identify and measure lipid yield of the sample. For example, identifying lipids for reactive oxygen species. It will be appreciated that a number of other suitable measurements can be included in the response parameter. The response parameter can then be used to provide a basis for adjusting the duty cycle, pulse frequency or intensity of the flux output conditions to optimise the temporal variations for sample growth.

In further embodiments, the processor of the assessment device may be in communication with a processor of the carbon capture system 100, such that the at least one light source of the illumination apparatus may be controlled to provide the optimised temporal variations in line with the outputted optimal temporal variations of flux output conditions identified by the assessment device. In other embodiments, the assessment device may be incorporated into the illumination apparatus (such as 350 of the system 100), such that the at least one light source of the illumination apparatus is controlled to identify and provide temporal variations in flux output, and a closed loop feedback system is used to adjust the illumination to find the optimal temporal variations for the algae being cultivated in the photobioreactor. In this embodiment, the sample plate is replaced with the plurality of trays which receive the algae solution.

### Method for Optimised Temporal Variations in Flux Output

There are also provided systems and methods for the carbon capture system to determine optimised temporal variations in flux output, and provide the optimised temporal variations in flux output.

In one embodiment, there is provided a method for determining optimal temporal variations in flux output for algal growth, including the steps of:
providing an algae solution on the at least one tray;
controlling that at least one light source of the illumination apparatus to emit light towards the at least one tray in accordance with flux output conditions, wherein the flux output conditions include at least a duty cycle and a pulse frequency;
exposing the at least one tray to the emitted light for a predefined time interval;
obtaining a first measurement of at least one response parameter related to the algae solution after it has been exposed to the emitted light for a defined time interval;
adjusting the flux output conditions based on the at least one response parameter;
obtaining a second measurement of at least one response parameter related to the algae solution after it has been exposed to emitted light having adjusted flux output conditions for a second defined time interval;
identifying an optimal set of flux output conditions from the at least one response parameter when the response parameter is determined to reach a threshold value;
outputting the optimal set of flux output conditions as an optimal temporal variation of flux output for the algae; and
controlling the at least one light source of the illumination apparatus in accordance with the optimal temporal variation of flux output.

In one embodiment, the assessment device may be in communication with the illumination apparatus (for example, 350) of the carbon capture system, such that a sample of algae provided in the assessment device is the algae being cultivated within the illumination apparatus, and the assessment device can determine the optimised temporal variations to be carried out by the illumination apparatus.

In one embodiment, a carbon capture system is configured to carry out the determination of optimal temporal variations in flux output, in which an algae solution is provided on the plurality of terraced illumination trays, and the light source of the illumination apparatus is controlled to emit light towards the trays in accordance with flux output conditions. The carbon capture system may be provided with at least one sensor to monitor a response parameter, and determine and adjust the flux output conditions accordingly to provide a closed loop feedback system for improved algae growth. When the optimal temporal variations in flux output are determined identified for the specific type of algae, the at least one light source of the illumination apparatus is controlled on the basis of the optimal temporal variation.

### Defouler

The carbon capture system may further include a defouler for cleaning the deposited algae biomass on the illumination tray in order to maintain the suspended and homogeneous algae solution. The degree of algae deposition on the illumination trays depends on the slope and hydraulic condition on the illumination trays. In general, higher slope will increase algae flow rate and reduce algae deposition. However, it comes with the reduction in number of illumination trays per footprint.

When algae is deposited on the trays of the illumination apparatus, the algae concentration is indicated by the turbidity value. A turbidity meter is used to observe the level of deposition and trigger the cleaning process. The deposited algae may be cleared using a centrifugal pump. The pump draws the algae solution from the algae solution holding tank and flushes the deposited biomass off the trays into the solution. The flushing cycles can be determined by the turbidity value set point or can be fixed by a specific time interval. For example, the time interval may be three cycles per day.

### CO₂ Sensor for Determining Carbon Removal

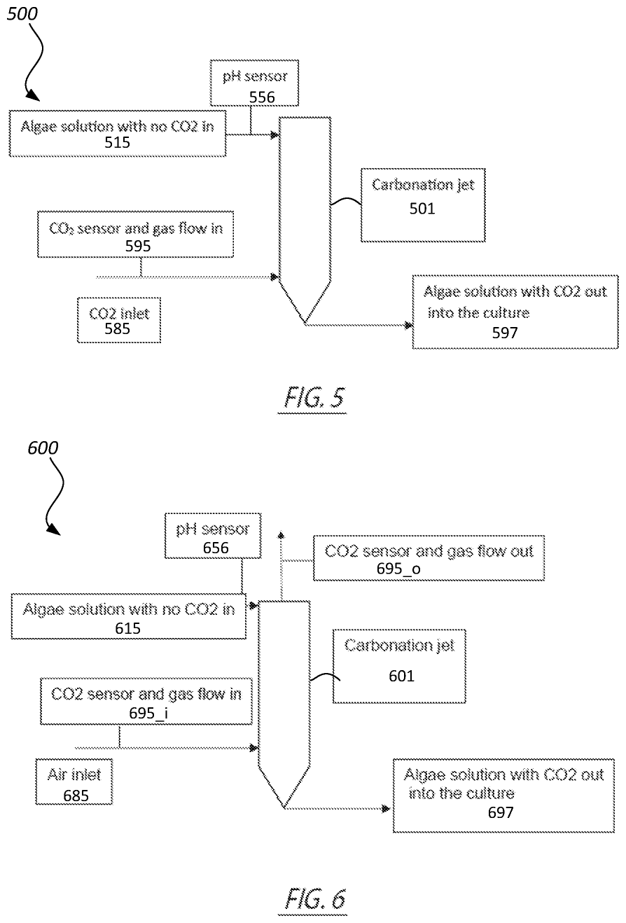

The carbon capture device may further include a CO₂ sensor. For CO₂ removal from concentrated sources, CO₂ concentration in the atmosphere and in the algae solution are measured by gas and dissolved CO₂ sensors, respectively. CO₂ injection into the algal solution of the carbon capture system is 100% CO₂×Mass flow (L/min)×dosing time (min). CO₂ is dosed via the carbonation device without any substantial gas loss. After dosing, the carbon capture system will take N minutes to consume the CO₂, where time N is measured between two dosing events, which is controlled by a pH set point in the algae solution. This pH set point is above 8.5 value to ensure all dissolved CO₂ in the ion form of HCO³⁻. This set point avoids any CO₂ loss from the algae solution back to the atmosphere. Thus, the total CO₂ dosing is equal to the total CO₂ capture, and the rate of carbon capture is total CO₂ dosing per N time. This configuration in the carbon capture system is shown in FIG. 5. In the example of FIG. 5 a system 500 includes a carbonation jet 501 which receives algae solution 515 with no CO₂. A pH sensor 556 detects pH of the solution 515. A CO₂ inlet 585 provides CO₂, which is sensed by a CO₂ sensor 595. The carbonation jet 501 outputs an algae solution 597 with CO₂. For CO₂ removal from atmosphere or flue gas, the CO₂ concentration and air flow rate are measured at the gas injection inlet of the carbonation device and a gas flow outlet disposed on the upper portion of the fluid vessel of the carbonation device. The gas flow outlet may be disposed on top of the fluid vessel of the carbonation device. The difference in the CO₂ concentration between the gas injection inlet and the gas flow outlet is the CO₂ absorbed in the algae solution. This configuration in the carbon capture system is shown in FIG. 6. In the example of FIG. 6 a system 600 includes a carbonation jet 601 which receives algae solution 615 with no CO₂. A pH sensor 656 detects pH of the solution 615. An air inlet 685 provides air, CO₂ levels of which are sensed by an inlet CO₂ sensor 695_i. A gas flow outlet sensor 695_o measures CO₂ concentration leaving the carbonation jet 601. The carbonation jet 601 outputs an algae solution 697 with CO₂.

A direct measurement of CO₂ removal by biomass generation may also be performed. CO₂ removal is mainly completed by acquisition into algae cells. Thus, a direct method to determine the amount of CO₂ removal is based on the production rate of algae biomass. For example, 1 kg of algae species of Scenedesmus sp has about 0.45 kg of carbon which is equivalent to 1.65 kg of CO₂.

### Photovoltaic Panel

In one embodiment, the carbon capture system includes at least one photovoltaic panel to provide power to the system. The photovoltaic panel is installed at a location above the terraced illumination apparatus. In some instances, the photovoltaic panel is installed on a roof of an enclosure in which the carbon capture system is housed. For example, the photovoltaic panel may be installed on the roof of a shipping container. In one example, where the carbon capture system is housed in a 20 ft standard shipping container, there are five photovoltaic panels with a capacity of 415 W. In total, from this configuration the carbon capture system is able to generate 12 to 16 KWh electricity per day under the assumption of receiving 6 to 8 hours of full sun (2 KW per hour energy production).

In some embodiments, the photovoltaic panel is mainly used to power the LED lights of the illumination apparatus. For example, the total energy usage for the LED light is 1.8 KWh. The utilisation of photovoltaic panels results in 20 times less CO₂ emissions than coal-powered energy. In some embodiments, the photovoltaic panel is used to power the at least one light source of the illumination apparatus. Additionally or alternatively, the photovoltaic panel is used to power the at least one secondary light source of the assessment device.

### Further Features

The carbon capture system of the present invention may include additional devices and systems. In one embodiment the carbon capture system includes at least one bioinformation sensor. The bioinformation sensor enables the system to obtain biological status information in order to inform optimal harvesting of the biomass. The bioinformation sensor may include at least one of a fluorometer, DO sensor, pH sensor, and a CO₂ sensor. Harvesting the biomass is completed at certain times or developmental stages of the algae that is informed by an array of sensors and/or algorithms. The harvesting is optimised for maximum CO₂ capture.

In another embodiment, the carbon capture system includes an automated nutrient dosing system. Nutrients are replenished based on calculated requirements or informed by an array of sensors that measure biological status or levels of nutrients available in the carbon capture system. The automated nutrient dosing system may control the supply of nutrients to the system based on measurements from the at least one bioinformation sensor.

The carbon capture system may further include a harvesting device, configured to harvest biomass from the carbon capture system. The harvesting deice may be configured to perform polymer flocculation. In one example, the harvester includes a rotating drum made of stainless steel mesh, an algae solution inlet, a clean water outlet, a dense algae biomass outlet, an algae collection trough, and a cleaning spray pump or air pump. The harvesting device can be operated continuously. In use, an algae solution is pumped into the centre of the rotating drum. Water is drained out by gravity and the algae biomass is retained. As the drum rotates, the retained biomass is moved upward. A spray pump or air pump is used to detach the algae biomass from the drum mesh, allowing it to fall by gravity to a collection trough at the middle of the drum. The mesh size of the rotating drum is adjustable in the range of 40 to 100 microns to match the target algae cell or aggregate sizes. The harvesting device can be used directly to remove or harvest multi-cell microalgae or applied in conjunction with a polymer flocculation. The polymer flocculation combines millions of individual algae cells into flocs.

While the embodiments herein reference algae, it will be appreciated that the carbon capture device can be used in applications relating to various plants, plant materials, bacteria and archaea, and that the devices can be used in various applications including, but not limited to, horticultural, agricultural, and aquacultural environments, as well as commercial glasshouses, hydroponics, tank-based seaweed, algae farms, and vertical farming production.

Although the invention has been described with reference to specific examples it will be appreciated by those skilled in the art that the invention may be embodied in many other forms.

1. A carbon capture system, including:
an illumination apparatus, the illumination apparatus, including:
at least one tray, the at least one tray including a substantially planar base and a peripheral rim around the base to form a receptacle;
a supporting frame configured to receive the at least one tray;
a light-dispersing layer disposed on a surface of the planar base to enable substantially uniform distribution of light through the planar base; and
at least one light source, positioned adjacent the planar base and configured to emit light toward the planar base such that at least a portion of the emitted light is dispersed by the light dispersing layer through the surface of the planar base.

2. The carbon capture system of claim 1, further including a carbonation device, the carbonation device including:
a fluid vessel;
a fluid supply system disposed on an upper portion of the fluid vessel to supply fluid to the fluid vessel;
a gas injection inlet disposed on a lower portion of the fluid vessel to supply gas to the fluid vessel;
at least one mixing element arranged within the fluid vessel and below the fluid supply system;
a diffuser arranged within the fluid vessel such that gas supplied through the gas injection inlet passes at least partially through the diffuser to produce a diffused gas, wherein the diffused gas and fluid mix within the fluid vessel to produce a mixed fluid; and
an outlet disposed on the fluid vessel to release the mixed fluid.

3. The carbon capture system of claim 1, wherein the illumination apparatus is configured to carry out a method for determining optimal temporal variations in flux output for algal growth, the method including the steps of:
providing an algae solution on the at least one tray;
controlling the at least one light source of the illumination apparatus to emit light towards the at least one tray in accordance with flux output conditions, wherein the flux output conditions include at least a duty cycle and a pulse frequency;
exposing the at least one tray to the emitted light for a predefined time interval;
obtaining a first measurement of at least one response parameter related to the algae solution after it has been exposed to the emitted light for a defined time interval;
adjusting the flux output conditions based on the at least one response parameter;
obtaining a second measurement of at least one response parameter related to the algae solution after it has been exposed to emitted light having adjusted flux output conditions for a second defined time interval;
identifying an optimal set of flux output conditions from the at least one response parameter when the response parameter is determined to reach a threshold value;
outputting the optimal set of flux output conditions as an optimal temporal variation of flux output for the algae solution; and
controlling the at least one light source of the illumination apparatus in accordance with the optimal temporal variation of flux output.

4. The carbon capture system of claim 1, further including a defouler.

5. The carbon capture system of claim 1, further including a carbon sensor.

6. The carbon capture system of claim 1, wherein CO₂ gas is supplied to the gas injection inlet of the carbonation device.

7. The carbon capture system of claim 1, further including at least one photovoltaic panel to provide power to the system.

8. The carbon capture system of claim 7, wherein the photovoltaic panel is installed at a location above the terraced illumination apparatus.

9. The carbon capture system of claim 7, wherein the photovoltaic panel is used to power the at least one light source of the illumination apparatus.

10. The carbon capture system of claim 1 further including an automated nutrient dosing system.

11. The carbon capture system of claim 1, further including at least one bioinformation sensor.

12. The carbon capture system of claim 11, wherein the bioinformation sensor includes at least one of a fluorometer, DO sensor, pH sensor, and a CO₂ sensor.

13. The carbon capture system of claim 11, wherein the automated nutrient dosing system controls the supply of nutrients to the system based on measurements from the at least one bioinformation sensor.

14. The carbon capture system of claim 1, further including a harvesting device, configured to harvest biomass from the carbon capture system.

15. The carbon capture system of claim 14, wherein the harvesting device is configured to perform polymer flocculation.
